(19) United States
(12) Patent Application Publication
(10) Pub. No.: US 2005/0096682 A1
(43) Pub. Date: May 5, 2005

(54) Vibratory blade device for body treatments

(75) Inventor: Steven J. Daffer, Edina, MN (US)

Correspondence Address: Brian D. Kaul;Westman, Champlin & Kelly, Suite 1600, 900 Second Avenue South, Minneapolis, MN, 55402-3319, US

(73) Assignee: Visibelle Derma Institute, Inc., Bloomington, MN (US)

(21) Appl. No.: 10/982,311

(22) Filed: Nov. 5, 2004

Related U.S. Application Data
(60) Provisional application No. 60/517,498, filed on Nov. 5, 2003.

(51) Int. Cl. A61B 17/32

(52) U.S. Cl. 606/169; 30/277.4

(57) ABSTRACT

A handheld vibratory blade device includes a main body, a vibrator, a blade receiver, and a blade member. The vibrator is supported by the main body and is configured to produce a vibration. The blade receiver is supported by the main body and is configured to receive the vibration produced by the vibrator. The blade member includes a distal end and a proximal end that is connected to the blade receiver.

The present application is based on and claims the benefit of U.S. provisional patent application Ser. No. 60/517,498, filed Nov. 5, 2003, the content of which is hereby incorporated by reference in its entirety.

### BACKGROUND OF THE INVENTION

The present invention generally relates to a handheld body treatment device and, more particularly, to a device that includes a vibrating blade member used to perform various body treatments.

Body treatments, such as cellulite reduction, body contouring, body sculpting, and emphatic drainage are very popular and effective ways to improve one's appearance. Such treatments include massaging targeted areas, particularly those that cannot be stimulated through exercise, to increase circulation and remove waste product. Additionally, various products can be applied to the skin for various purposes including increasing the effectiveness of a body treatment.

There is a continuous demand for improvements to body treatments including new body treatment methods utilizing specialized tools for enhancing the effectiveness of the body treatments while reducing the effort required to perform them.

### SUMMARY OF THE INVENTION

The present invention is directed to a handheld vibratory blade device that is designed to be used to perform various body treatments. The device includes a main body, a vibrator, a blade receiver, and a blade member. The vibrator is supported by the main body and is configured to produce a vibration. The blade receiver is supported by the main body and is configured to receive the vibration produced by the vibrator. The blade member includes a distal end and a proximal end that is connected to the blade receiver.

Additional embodiments of the vibratory blade device include a base unit. The base unit includes a motor and a cable having a first end that is rotatably driven by the motor. The vibrator includes a transducing component that is configured to produce the vibration in response to a rotation of the cable.

Other features and benefits that characterize embodiments of the present invention will be apparent upon reading the following detailed description and review of the associated drawings.

### BRIEF DESCRIPTION OF THE DRAWINGS

FIG. 1 is a simplified diagram of a vibratory blade device in accordance with embodiments of the invention.

FIG. 2 is a cross-sectional view of a portion of the vibratory blade device in accordance with embodiments of the invention.

FIG. 3 is a magnified view of the locking member of a blade receiver shown in FIG. 2.

FIGS. 4-7 are front views of blade members in accordance with various embodiments of the invention.

FIGS. 8 and 9 are side views of the distal end of blade members that include a textured surface in accordance with embodiments of the invention.

FIG. 10 is a simplified side view of the blade member end of a vibratory blade device that includes a light source in accordance with embodiments of the invention.

FIG. 11 is a simplified view of the blade member end of a vibratory blade device that includes a lotion dispensing component in accordance with various embodiments of the invention.

FIG. 12 is a simplified view of a blade member end of a vibratory blade device that includes a gas dispensing component in accordance with various embodiments of the invention.

### DETAILED DESCRIPTION OF THE PREFERRED EMBODIMENTS

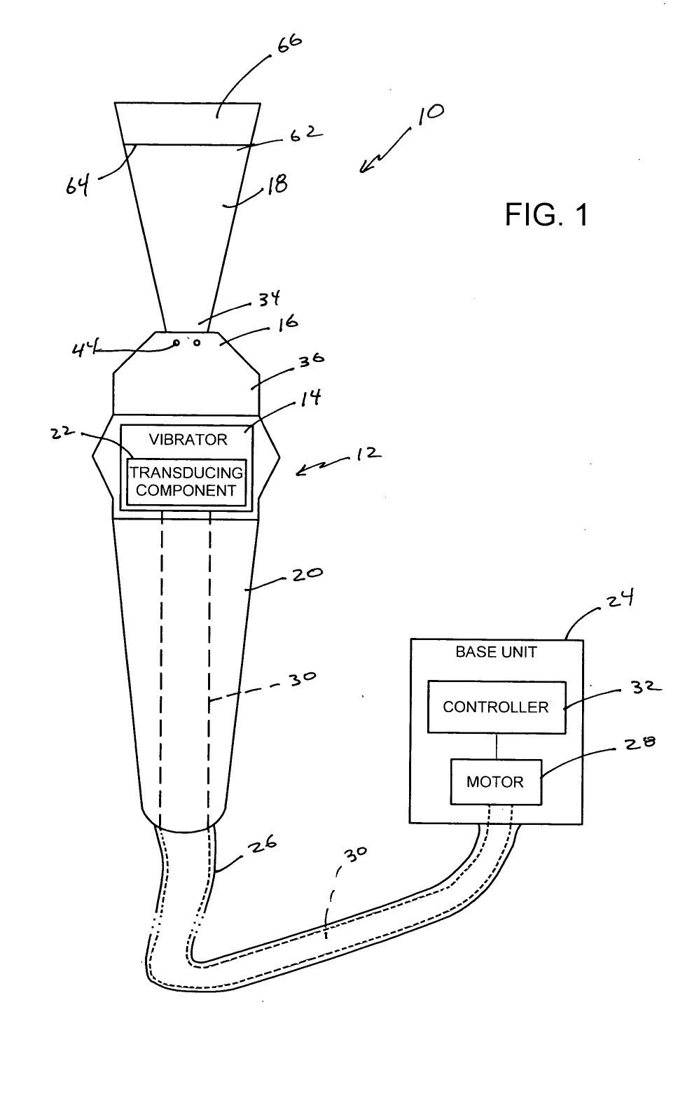

FIG. 1 is a simplified diagram of a vibratory blade device 10 for applying products to the skin and for performing body treatments. The body treatments that can be performed by the device 10 include, for example, cellulite reduction, body contouring, body sculpting, lymphatic drainage, lotion and cosmetic applications and other body treatments. Additionally, the vibratory blade device 10 can be used to provide controlled spreading of sub-dermal injections, tissue stimulation treatments such as for collagen, elastin, circulatory, and edema, and skin surface treatments such as exfoliation, abrasion or esthetic removal.

Vibratory blade device 10 generally includes a main body 12, a vibrator 14, a blade receiver 16, and a blade member 18. The main body 12 provides support for the components of the device 10. In accordance with one embodiment of the invention, the main body 12 includes a handle 20.

The vibrator 14 is supported by the main body 12 and is generally configured to produce a vibration in accordance with conventional methods. The vibration can include vibratory motions that are parallel, perpendicular, and/or aligned with a given axis. Additionally, the frequency of the vibratory motion is preferably variable between 1-60 hertz or more.

One embodiment of vibrator 14 includes a transducing component 22 that produces the vibration in response to a motorized input, such as a rotating cable, as will be discussed below. Transducing component 22 can include a cam mechanism that generates the desired vibration, or a rotatable eccentric member whose rotation produces the vibration. Vibrator 14 can take on other forms and use different methods to generate the desired vibration.

The motorized input to the transducing component 22 can be produced by a motor supported by the main body 12 and powered by an on-board power supply (i.e., batteries), or powered by external source. In accordance with a preferred embodiment of the invention, the vibratory blade device 10 includes a separate base unit 24 that is configured to produce the motorized input. Tubing 26 connects to the main body 12 at, for example, handle 20, as shown in FIG. 1. The base unit 24 includes a motor 28 and a cable 30 that is rotatably driven by the motor 28 under control of a controller 32. An end of the cable 30 connects to the vibrator 14 to provide the motorized input for the transducing component 22.

The blade receiver 16 is configured to receive a proximal end 34 of the blade member 18 and the vibration from the vibrator 14. One embodiment of the blade receiver 16 includes a base 36. The base 36 and the blade member 18 are preferably formed of metal, but can be formed of other materials, such as plastic, if desired.

The vibration produced by vibrator 14 is received by the base 36 and the blade member 18 that is connected to the blade receiver 16. The resultant vibration of the blade member 18 can be used to assist in the performance of various body treatments. For example, the vibration of the blade member 18 can assist in the opening of pores of the skin to provide better penetration of products that are applied to the skin, such as lotions, ointments, cosmetics, etc. Additionally, the vibration of the blade member 18 can assist in exfoliating the skin and sculpting the body when the blade member 18 is configured for those purposes, as will be explained below in greater detail.

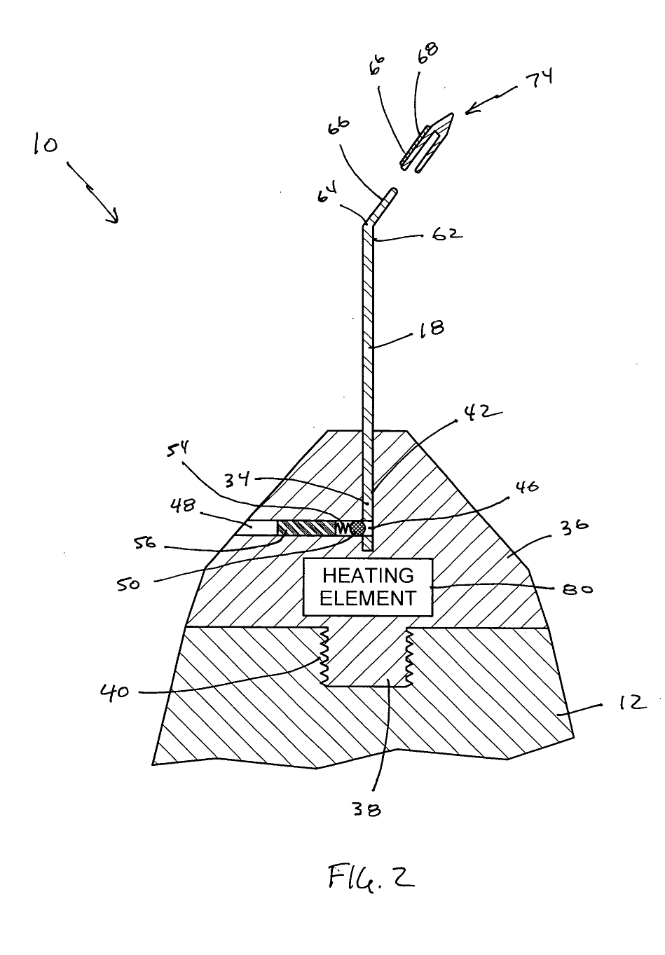

The base 36 can be formed integral with the main body 12 or separate therefrom. When formed separately from the main body 12, the base 36 preferably attaches to main body 12 such that it is prevented from rotating relative thereto when fully mounted. In accordance with one embodiment of the invention, the base 36 includes a screw member 38 that can be received by a threaded socket 40 of the main body 12 to secure the base 36 thereto, as shown in the cross-sectional view of FIG. 2. Other methods of attachment can also be used.

As mentioned above, the blade receiver 16 is configured to receive the blade member 18 and connect it to the main body 12. In accordance with one embodiment of the invention, the blade receiver 16 includes a slot 42 that receives the proximal end 34 of the blade member 18. Preferably, the blade receiver 16 and the proximal end 34 of the blade member 18 include cooperating connecting members that allow for quick attachment of the blade member 18 to the main body i2 or the base 36 by hand. Alternatively, screws (e.g., screws 44 in FIG. 1), latching mechanisms, and other attaching means can be used to removably secure the blade member 18 to base 36.

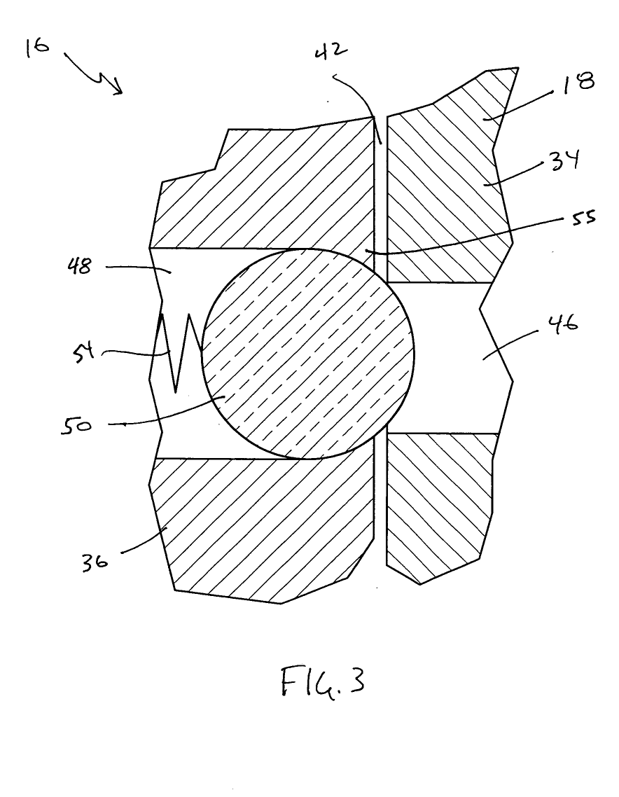

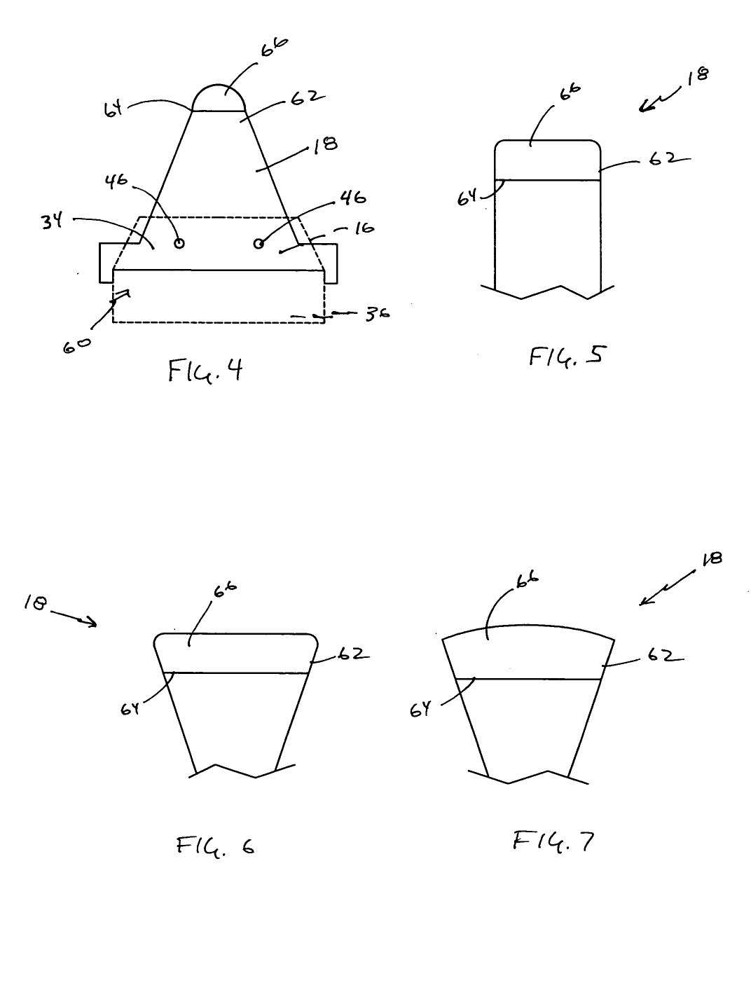

FIG. 2 illustrates one embodiment of the blade receiver 16. A magnified view of this embodiment of the blade receiver 16 is provided in FIG. 3. In general, the blade member 18 includes one or more apertures 46 at the proximal end 34, as shown in FIG. 4. The blade receiver 16 includes corresponding bores 48 in the base 36 that correspond to each of the apertures 46 in the blade member 18.

Each of the bores 48 includes a movable locking member 50 that is configured to move between retracted and locking positions. In accordance with one embodiment of the invention, the locking member 50 includes a ball bearing, as shown in FIGS. 2 and 3, but other suitable components can be used as well. When the locking member 50 is in the retracted position, the blade member 18 can be received by blade receiver 16, such as within the slot 42. When the blade member 18 is fully received within the blade receiver 16 such that the aperture 46 of the blade member 18 aligns with the corresponding bore 48 of the blade receiver 16, the locking member 50 moves to the locking position, in which at least a portion of the locking member 50 extends into the corresponding aperture 46 of the blade member 18, as shown in FIG. 3, thereby removably securing the blade member 18 to the main body 12.

In accordance with one embodiment of the invention, the locking member 50 is biased by a spring 54, or in another suitable manner, toward the locking position. Thus, in the exemplary block receiver 16 shown in FIGS. 2 and 3, the locking member 50 is biased toward the slot 42.

The locking member 50 is preferably prevented from extending into the slot 42 an amount that would prevent the full insertion of the proximal end 34 of the blade member 18 into the slot 42. In accordance with one embodiment of the invention, the locking member 50 is maintained in bore 48 by a shoulder portion 55 (FIG. 3) of the bore 48 that is adjacent the slot 42. The spring 54, used to bias the locking member 50 toward the locking position, is held in compression within the bore 48 by a screw 56 (FIG. 2) or other suitable component.

With this configuration, as the proximal end 34 of the blade member 18 is inserted in blade receiver 16, the locking member 50 (i.e., such as a ball bearing) engages the bottom edge of the blade member 18, which counteracts the biasing force applied by the spring 54 and moves the locking member toward the retracted position to allow the blade member 18 to continue to slide into the slot 42. When the aperture 46 of the blade member 18 aligns with the bore 48, the locking member 50 is moved into the locking position by the spring 54 such that a portion of the locking member 50 extends into the aperture 46, as shown in FIG. 3. In this manner, the blade member 18 is sufficiently secured to use the vibratory blade device 10 while allowing for the removal of the blade member 18 from the blade receiver 16 by hand. This allows for quick changes of the blade members 18.

Alternatively, the position of the locking member 50 can be controlled by a suitable finger-actuated mechanism. The locking member 50 can then extend through the aperture 46 of the blade member 18. Removal of the blade member 18 preferably requires actuation of the mechanism to place the locking member 50 in the retracted position, in which the locking member no longer extends through the aperture 46 of the blade member 18.

Another embodiment of the blade member 18 includes an alignment feature 60 that aligns the blade member 18 to the blade receiver 16 on the base 36, as shown in FIG. 4. In accordance with one embodiment of the invention, the alignment feature 60 is in the form of a notch in the proximal end 34 of the blade member 18. One embodiment of the notch 60 extends around the base 36 (shown in dashed lines) to align itself therewith. Other types of alignment features can also be used to ensure the proper orientation of the blade member 18 relative to the base 36.

The blade member 18 can take on various shapes and have various features at distal end 52 to facilitate performing different body treatments, as illustrated in FIGS. 1 and 4-7. For instance, the blade member 18, shown in FIG. 4, includes a small (e.g., thumb-sized) half circle at a distal end 62 that is sized to allow for treatment of the skin adjacent the eye socket and along the bridge of the nose. During use, an index finger of the operator can apply pressure directly to the distal end 62 during the skin or body treatment. Additionally, the blade member 18 have a rectangular shape (FIG. 5), a triangular shape (FIG. 6), a conical shape (FIG. 7), have a flat edge at the distal end 62 (FIGS. 1, 5 and 6), a curved edge at the distal end 62 (FIGS. 4 and 7), be wider at the distal end 62 than the proximal end 34 (FIG. 4), be narrower at the distal end 62 than the proximal end 34 (FIGS. 1, 6 and 7), or be any other desired shape.

In accordance with one embodiment of the invention, the blade member 18 includes a bend 64 (shown best in FIG. 2), of preferably approximately 450, that defines a body engaging surface 66 that is designed to engage the body or skin of a subject during body treatments. Accordingly, the bend 64 causes the body engaging surface 66 at the distal end 62 to be non-coplanar with the proximal end 34 of the blade member 18. The bend 64 allows the operator to maintain a more comfortable hand position while performing the body treatment including spreading of a product over the skin.

The flex of the blade member 18 can be set in accordance with the desired application. The length and thickness of the blade member 18, as well as the material chosen to form the blade member 18 can be adjusted to provide the desired flex. For instance, a flexible blade member 18 can be used when low pressure is desired to be applied at the skin engaging surface 66. Alternatively, a stiff blade member 18 is desired for more rugged body treatments, such as body sculpting.

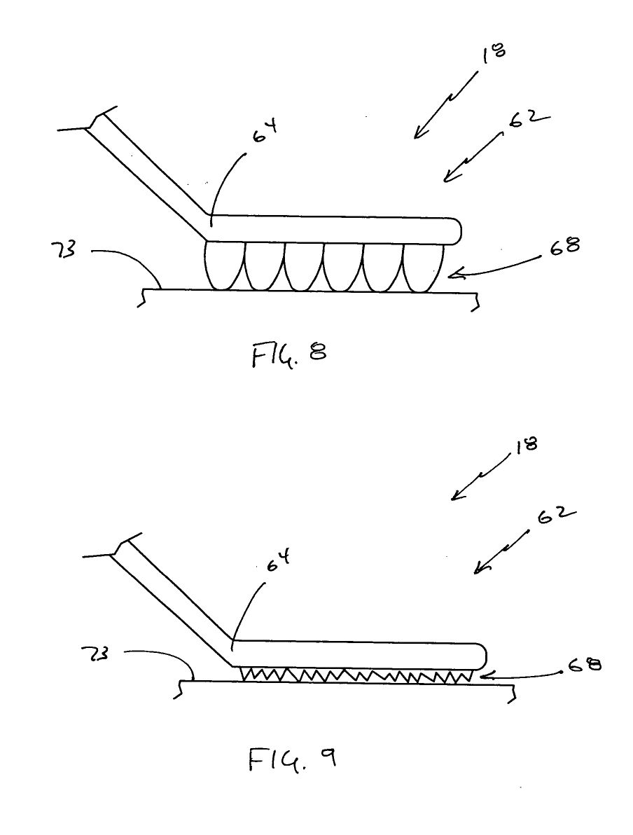

In accordance with one embodiment of the invention, the body engaging surface 66 includes a textured surface 68 having massaging bumps or protrusions 70 (FIG. 8), or an abrasive layer of material 72 (FIG. 9), such as a diamond coating. Bumps 70 can be used for body sculpting or massaging purposes, whereas abrasive layer 72, can be used to exfoliate the skin 73, for example.

In accordance with another embodiment of the invention, the device 10 includes a sleeve member 74, shown exploded from the blade member 18 in FIG. 2, that receives the distal end 62 of the blade member 18 and forms a new skin engaging surface 66. The sleeve member 74 can be configured to receive a larger portion of the blade member 18 beyond the skin engaging surface 66 of the blade member 18 if desired, particularly when there is no bend 64 in the blade member 18.

The sleeve member 74 is preferably a disposable member that is formed of plastic, rubber, or other suitable material. The sleeve member 74 can also include a textured surface 68 in the form of bumps, an abrasive layer, or other texture, as described above with respect to FIGS. 8 and 9. As a result, different textured surfaces 68 can be quickly attached to a single blade member 18 by using multiple sleeve members 74.

Additionally, the sleeve member 74 can take on various shapes and facilitate different body treatments. For example, a first sleeve member 74 can be attached to the distal end 62 of the blade member 18 to facilitate even spreading of a lotion or cosmetic, then another sleeve member 74 can replace the first sleeve member to facilitate working the lotion or cosmetic into the skin, without having to remove the blade member 18 from the blade receiver 16. As a result, it may not be necessary to utilize multiple blade members 18 with the device 10. Therefore, in accordance with one embodiment of the invention, the blade member 18 and blade receiver are integrally formed thereby making the blade member 18 non-removable from the blade receiver 16.

One embodiment of the vibratory blade device 10 includes a heating element 80 (FIG. 2) that is configured to generate heat that is conducted to the blade member 18 to facilitate more comfortable body treatments. The heating element 80 can be positioned within the base 36 (shown), or provided elsewhere in the main body 12. The conduction of the heat generated by the heating element 80 is facilitated by the base 36, and the blade member 18 being formed of metal. The heating element 80 can receive power from the base unit 24 (FIG. 1), a battery contained in the main body 12, or other suitable source.

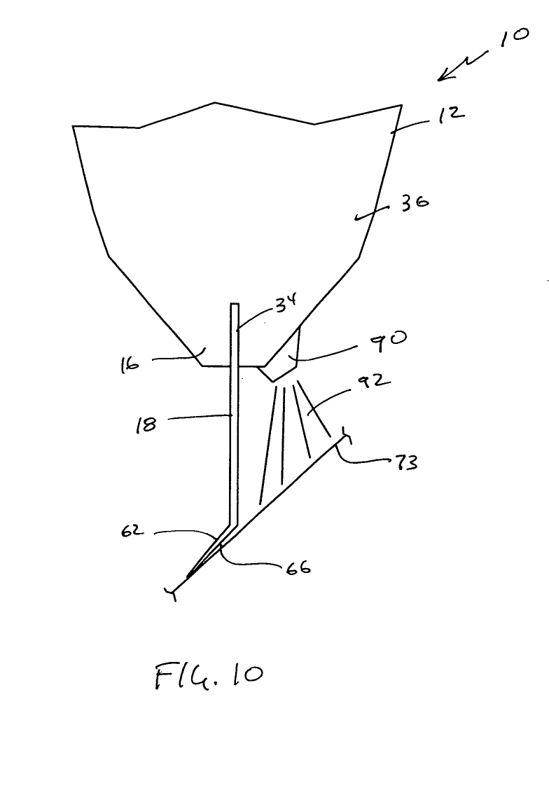

In accordance with another embodiment of the invention, the vibratory blade device 10 includes a light source 90, as shown in FIG. 10. The light source 90 is supported by the main body 12 adjacent the blade receiver 16 and is configured to transmit light 92 generally toward an area of the body 73 being treated that is adjacent the distal end 62 of the blade member 18. The light source 90 can include a light emitting diode (LED), a laser, or other suitable light source that can be used to provide a desired light treatment to the skin or otherwise illuminate the surface being treated. The light source 90 can receive power from the base unit 24 (FIG. 1), a battery contained in the main body 12, or other suitable source.

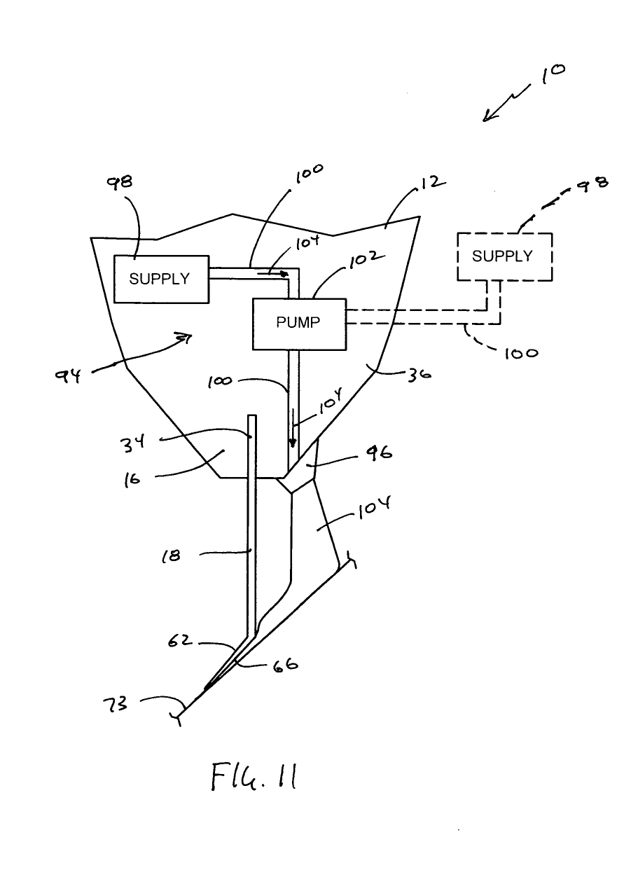

In accordance with another embodiment of the invention, the vibratory blade device 10 includes a lotion dispensing component 94, shown in FIG. 11, that is configured to dispense a body treatment lotion, an ointment, a cosmetic or other fluid to the body or skin 73. One embodiment of the lotion dispensing component 94 includes a nozzle 96 supported by the main body 12, a supply of body treatment lotion 98, a fluid flow path 100, and a pump 102.

The fluid flow path 100 connects the supply 98 to the nozzle 96. The pump 102 is configured to deliver lotion 104 from the supply 98 to the nozzle 96 and discharge the lotion through the nozzle 96 and onto the area of the body 73 being treated. Accordingly, the pump 102 can be positioned inline with the fluid flow path as shown, or be used to pressurize the supply 98. The supply 98 can be located within the main body 12 of the vibratory blade device 10, or remotely from the main body 12 (as shown in dashed lines). Additionally, the pump 102 can be located remotely from the main body 12.

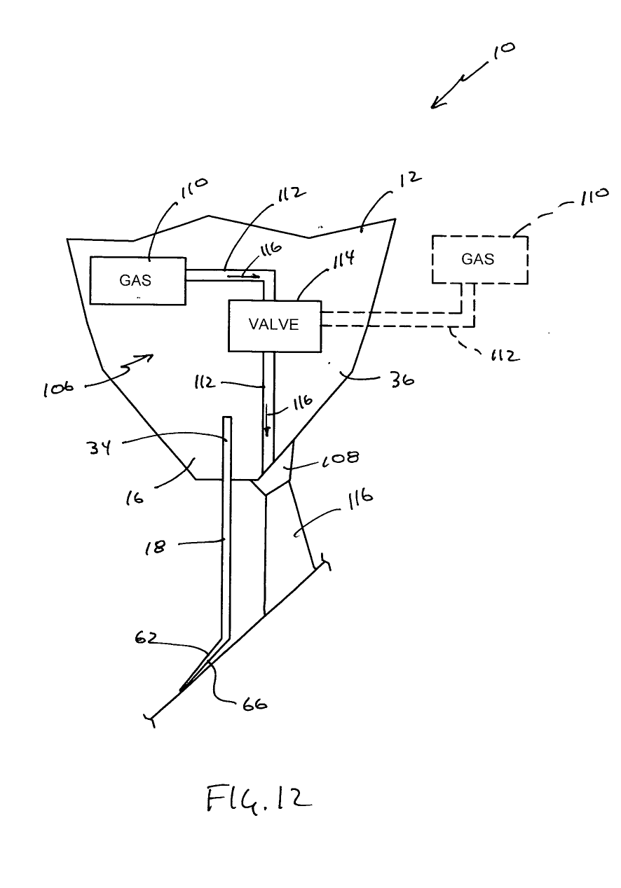

In accordance with another embodiment of the invention, the vibratory blade device 10 includes a gas dispensing component 106, shown in FIG. 12, that is configured to dispense a gas, such as oxygen, to the skin 73 while the blade member 18 is drawn or pushed across the skin 73. One embodiment of the gas dispensing component 106 includes a nozzle 108 supported by the main body 12, a supply of pressurized gas 110, a fluid flow path 112, and a valve 114. The valve 114 includes an opened position in which the fluid flow path 112 is opened between the supply 110 and the nozzle 108 to allow gas 116 to flow there between and through the nozzle 108 and onto the area of the body 73 being treated, and a closed position in which the fluid flow path 112 is closed between the nozzle 108 and the supply 110. The supply 110 can be located within the main body 12 of the vibratory blade device 10, or remotely from the main body 12 (as shown in dashed lines). Additionally, the valve 114 can be located remotely from the main body 12.

Although the present invention has been described with reference to preferred embodiments, workers skilled in the art will recognize that changes may be made in form and detail without departing from the spirit and scope of the invention. For example, the nozzles used to dispense gas or lotion, and the light source discussed above can be connected to other parts of the device 10, such as the blade member 18.

1. A handheld vibratory blade device comprising:
a main body including a handle;
a vibrator supported by the main body and configured to produce a vibration;
a blade receiver supported by the main body and configured to receive the vibration produced by the vibrator; and
a blade member having a distal end and a proximal end connected to the blade receiver.

2. The device of claim 1 including a base unit comprising a motor and a cable having a first end rotatably driven by the motor, wherein the vibrator includes a transducing component configured to produce the vibration in response to a rotation of the cable.

3. The device of claim 1, wherein:
the blade includes an aperture at the proximal end; and
the blade receiver includes a slot configured to receive the proximal end, and a locking member that extends at least partially through the aperture to secure the blade in the blade receiver.

4. The device of claim 3, wherein the locking member includes a retracted position in which the proximal end of the blade is receivable within the slot, and a locking position in which the locking member at least partially extends through the aperture.

5. The device of claim 4, wherein the locking member is biased toward the locking position.

6. The device of claim 4, wherein the locking member includes a ball bearing contained in a bore of the blade receiver and biased toward the locking position.

7. The device of claim 1, wherein the proximal end of the blade member includes a notch that is configured to align the blade member with the blade receiver.

8. The device of claim 1, wherein the blade member is formed of a sheet of material including a material selected from a group consisting of metal and plastic.

9. The device of claim 1, wherein the blade member includes a bend between the proximal and distal ends thereby forming a body engaging section of the blade member at the distal end that is non-coplanar to the proximal end of the blade member.

10. The device of claim 1, wherein the distal end of the blade member includes a curved edge.

11. The device of claim 1, wherein the distal end of the blade member includes a flat edge.

12. The device of claim 1, wherein the distal end is wider than the proximal end.

13. The device of claim 1, wherein the proximal end is wider than the distal end.

14. The device of claim 2, wherein the second end of the cable extends through the handle.

15. The device of claim 1 including a heat source supported by the main body and configured to generate heat that is conducted to the blade member.

16. The device of claim 1 including a light source supported by the main body and configured to transmit light toward an area that is adjacent to the distal end of the blade member.

17. The device of claim 1 including a lotion dispensing component comprising:
a nozzle supported by the main body;
a supply of body treatment lotion;
a fluid flow path connecting the supply to the nozzle; and
a pump configured to deliver the lotion to the nozzle and discharge the lotion through the nozzle.

18. The device of claim 1 including a gas dispensing component comprising:
a nozzle supported by the main body;
a supply of pressurized gas; and
a valve having an opened position in which a fluid flow path is opened between the supply and the nozzle, and a closed position in which the fluid flow path is closed between the nozzle and the supply.

19. A handheld vibratory blade device comprising:
a base unit having a motor and a cable rotatably driven by the motor at a proximal end;
a main body including a handle;
a vibrator supported by the main body and configured to produce a vibration in response to a rotation of the cable;
a blade receiver supported by the main body and configured to receive the vibration produced by the vibrator; and
a blade member having a distal end, and a proximal end connected to the blade receiver.

20. The device of claim 19, wherein the vibrator includes a transducing component configured to produce the vibration in response to the rotation of the cable.

21. The device of claim 19, wherein:
the blade includes an aperture at the proximal end; and
the blade receiver includes a slot configured to receive the proximal end of the blade member and a locking member that extends at least partially through the aperture to connect the blade member to the blade receiver.

22. The device of claim 21, wherein the locking member includes a retracted position in which the proximal end of the blade is receivable within the slot, and a locking position in which the locking member at least partially extends through the aperture.

23. The device of claim 22, wherein the locking member includes a ball bearing contained in a bore of the blade receiver and biased toward the locking position.

24. The device of claim 19, wherein the proximal end of the blade member includes a notch that is configured to align the blade member with the blade receiver.

25. The device of claim 19, wherein the blade member is formed of a sheet of material including material from a group consisting of metal and plastic.

26. The device of claim 19, wherein the blade member includes a bend between the proximal and distal ends thereby forming a body engaging section of the blade member at the distal end that is non-coplanar to the proximal end of the blade member.

27. The device of claim 19, wherein the distal end of the blade member includes an edge having a shape selected from a group consisting of curved and flat.

28. The device of claim 19, wherein the distal end of the blade member is wider than the proximal end of the blade member.

29. The device of claim 19, wherein the proximal end of the blade member is wider than the distal end of the blade member.

30. The device of claim 19 including a heat source supported by the main body and configured to generate heat that is conducted through the blade member.

31. The device of claim 19 including a light source supported by the main body and configured to transmit light toward an area that is adjacent to the distal end of the blade member.

32. The device of claim 19 including a lotion dispensing component comprising:
a nozzle supported by the main body;
a supply of body treatment lotion;
a fluid flow path connecting the supply to the nozzle; and
a pump configured to deliver the lotion to the nozzle and discharge the lotion through the nozzle.

33. The device of claim 19 including a gas dispensing component comprising:
a nozzle supported by the main body;
a supply of pressurized gas; and
a valve having an opened position in which a fluid flow path is opened between the supply and the nozzle, and a closed position in which the fluid flow path is closed between the nozzle and the supply.
